(19) United States
(12) Patent Application Publication
(10) Pub. No.: US 2023/0257819 A1
(43) Pub. Date: Aug. 17, 2023

(54) APPLICATION OF BIOMARKERS IN PREPARING DIAGNOSTIC TOOLS FOR COMBINED ALLERGIC RHINITIS AND ASTHMA SYNDROME

(71) Applicant: The Affiliated Changzhou Second People's Hospital of Nanjing Medical University, Changzhou City (CN)

(72) Inventor: Qian ZHANG, Changzhou (CN)

(21) Appl. No.: 18/148,062

(22) Filed: Dec. 29, 2022

(30) Foreign Application Priority Data
Feb. 16, 2022 (CN) 2022101410277

(51) Int. Cl. C12Q 1/6883

(52) U.S. Cl. CPC C12Q 1/6883; C12Q 2600/118; C12Q 2600/158; C12Q 2600/178

(57) ABSTRACT

Disclosed is an application of biomarkers in preparing diagnostic tools for combined allergic rhinitis and asthma syndrome (CARAS), including circ_0070934, MGAT3 and miR-199a-5p. The biomarker of the present application can be effectively used for the diagnosis of CARAS, and when circ_0070934, miR-199a-5p and MGAT3 are combined, the diagnostic effectiveness of CARAS is the highest.

### CROSS-REFERENCE TO RELATED APPLICATIONS

This application claims priority to Chinese Patent Application No. 202210141027.7, filed on Feb. 16, 2022, the contents of which are hereby incorporated by reference.

### TECHNICAL FIELD

The present application belongs to the technical field of biomedicine, and particularly relates to an application of biomarkers in preparing diagnostic tools for combined allergic rhinitis and asthma syndrome (CARAS).

### STATEMENT REGARDING SEQUENCE LISTING

The sequence listing associated with this application is provided in text format in lieu of a paper copy and is hereby incorporated by reference into the specification. The name of the XML file containing the sequence listing is 65358-updated SL. The XML file is 9,136 bytes; was created on Feb. 16, 2023; contains no new matter; and is being submitted electronically via EFS-Web.

### BACKGROUND

Bronchial asthma (or asthma) is a recurrent chronic airway disease featured by chronic airway inflammation, airway hyperresponsiveness, reversible airflow limitation and airway remodeling, also manifested by recurrent episodes of wheezing, shortness of breath, chest tightness, cough and other respiratory symptoms. Allergic rhinitis and asthma are inflammatory diseases of the upper and lower respiratory tracts, respectively, with similar etiology, immunology and mechanisms of occurrence. Therefore, a new medical diagnostic term, combined allergic rhinitis and asthma syndrome (CARAS), is proposed in recent years based on the concept of “same airway, same disease”, which refers to the simultaneous occurrence of clinical or subclinical upper respiratory allergy (allergic rhinitis) and lower respiratory allergic symptoms (asthma).

Non-coding RNAs (ncRNAs) refer to RNA molecules that are not translated into protein in transcriptome, including long non-coding RNAs (lncRNAs), micro RNAs (miRNAs), circular RNAs (circRNAs), etc.; ncRNAs play an important role in the occurrence and development of allergic diseases (including allergic rhinitis and asthma), participating in important processes such as airway inflammation response, airway remodeling, airway hyperresponsiveness, epithelial differentiation, mucus production, etc., and are expected to be developed into biomarkers for disease diagnosis and therapeutic targets.

### SUMMARY

The present application provides biomarkers in diagnosing combined allergic rhinitis and asthma syndrome (CARAS), and such an objective is achieved by the following technical schemes:

one aspect of the present application provides biomarkers for diagnosing CARAS, including circ_0070934, MGAT3, and miR-199a-5p.

The term “diagnosis” as used herein is defined as an identification or classification of a molecular or pathological state, disease or condition. For example, “diagnosis” may refer to the identification of the risk of developing CARAS by the tissue/organ involved (e.g., CARAS related issue/organ), or by molecular characteristics (e.g., characterized by the expression of a specific gene or one or a combination of proteins encoded by the gene). The term “diagnosis” includes determining whether a subject has the CARAS, and determining the risk of the subject developing the CARAS. As is well known to those skilled in the art, the steps of correlating biomarker levels with a certain Likelihood or risk can be performed and implemented in different ways.

The term “and/or” as used herein in phrases such as “A and/or B” is intended to include both A and B; A or B; A (alone); and B (alone). Similarly, the term “and/or” as used in phrases such as “A, B, and/or C” is intended to cover each of the following embodiments: A, B, and C; A, B, or C; A or C; A or B; B or C; A and C; A and B; B and C; A (alone); B (alone); and C (alone).

The term “biomarker” refers to a biomolecule that is present in an individual at different concentrations and can be used to predict an individual's disease state. Biomarkers may include, but are not limited to, nucleic acids, proteins, and variants and fragments thereof. Biomarkers may be DNA comprising all or part of a nucleic acid sequence encoding the biomarker or a complement of such a sequence. Biomarker nucleic acids that can be used in the present application are considered to include DNA and RNA comprising all or part of the sequence of any target nucleic acid sequence.

In one embodiment, at least one of the genes described is compared to a reference level of at least one of the genes described, so as to determine whether the individual has/suffers CARAS.

In a preferred embodiment, the reference level is a level determined by measuring at least one reference biological sample (e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41 42, 43, 44, 45, 46, 47, 48, 49, 50, 100, 150, 200, 250, 300, 400, 500 or 1,000 reference biological samples) isolated from at least one (healthy) subject not suffering from CARAS (control) (e.g. isolated from at least 1, 2, 3, 4, 5, 6, 7, 8, 9, 10, 11, 12, 13, 14, 15, 16, 17, 18, 19, 20, 21, 22, 23, 24, 25, 26 27, 28, 29, 30, 31, 32, 33, 34, 35, 36, 37, 38, 39, 40, 41, 42, 43, 44, 45, 46, 47, 48, 49, 50, 100, 150, 200, 250, 300, 400, 500 or 1,000 (control) subjects without CARAS). The at least one subject described as not suffering from CARAS may be considered healthy relative to a person suffering from CARAS.

In a preferred embodiment, the expression level of miR-199a-5p is upregulated in subjects with CARAS and the expression levels of circ_0070934, MGAT3 are downregulated in subjects with CARAS compared to that of normal control (reference level).

The terms “expression level”, “level of expression”, and “amount of expression” as used herein refer to the amount of polynucleotide or amino acid products or proteins in a biological sample. “Expression” usually refers to a process by which the information encoded by a gene is translated into a structure that exists and functions in a cell. Thus, “expression” of a biomarker as used herein refers to transcription into polynucleotides, translation into proteins, or even post-translational modifications of proteins. Transcribed polynucleotides, translated proteins, or post-translationally modified segments of proteins are also considered as expressed, whether they originate from transcripts generated by selective splicing or degraded transcripts, or from post-translational processing of proteins (e.g., by protein hydrolysis), “Expressed genes” include those that are transcribed as polynucleotides (e.g., mRNA) and then translated into proteins, as well as those that are transcribed as RNA but not translated into proteins (e.g., transfer RNA and ribosomal RNA).

Another aspect of the present application provides an application of reagents for detecting the biomarkers described in an aspect of the present application in a sample for preparing diagnostic tools of CARAS.

Optionally, the reagents include reagents for detecting the biomarkers in terms of mRNA expression levels, and reagents for detecting the biomarkers in terms of protein expression levels.

Optionally, the reagents for detecting the biomarkers in terms of mRNA expression levels adopt the following methods: polymerase chain reaction (PCR)-based detection method, Southern hybridization method, Northern hybridization method, dot hybridization method, fluorescence in-situ hybridization method, DNA microarray method, allele-specific oligonucleotide (ASO) method, high-throughput sequencing platform method and chip method.

Optionally, the reagents for detecting the protein expression levels of the biomarkers adopt the following methods: protein blotting (Western Blot method), enzyme-linked immunosorbent assay (ELISA), radioimmunoassay (RIA), sandwich assay, immunohistochemical staining, mass spectrometry, immunoprecipitation analysis, complement fixation analysis, fluorescence activated cell sorting (FACS) method and protein chip method.

Optionally, the reagents include:

primer pairs, probes or antisense nucleotides specifically combined with genes of the biomarkers; or antibodies, interacting proteins, ligands, nanoparticles or aptamers specifically bound to proteins or peptide fragments of the biomarkers.

The term “primer” refers to a fragment that recognizes a target gene sequence, including forward and reverse primer pairs, preferably primer pairs that provide specific and sensitive analysis results. The primers have nucleic acid sequences that are inconsistent with non-target sequences present in a sample and confer high specificity when they are primers that only amplify target gene sequences containing complementary primer binding sites and do not induce non-specific amplification.

The term “probe” refers to a substance that can specifically bind to a target substance to be detected in the sample, and refers to a substance that can confirm the existence of the target substance in the sample through the above-mentioned binding specificity. The probes shall be of a type commonly used in the art with no limitation and, preferably, include peptide nucleic acid (PNA), locked nucleic acid (LNA), peptide, polypeptide, protein, ribonucleic acid or deoxyribonucleic acid, most preferably peptide nucleic acid. More specifically, the above probes are biological substances, including those derived from or similar to biological materials or manufactured in vitro, and may include, for example, enzymes, proteins, antibodies, microorganisms, plant and animal cells and organs, nerve cells, deoxyribonucleic acids and ribonucleic acids, deoxyribonucleic acids including complementary deoxyribonucleic acids (cDNA), genomic deoxyribonucleic acids, oligonucleotides, ribonucleic acids including genomic RNA, messenger RNA, oligonucleotides, and examples of proteins include antibodies, antigens, enzymes, peptides, etc.

The term “antisense” as used in the present application refers to the hybridization of antisense oligomers to target sequences in ribonucleic acid by base complementary pairing, which typically allows the formation of messenger ribonucleic acid and ribonucleic acid: oligo-heteroduplexes, oligomers with sequences of nucleotide bases and inter-subunit backbones in the target sequence. Oligomers may have precise sequence complementarily or near-complementarity with respect to the target sequence.

The term “antibody” refers to a substance that specifically binds to an antigen to cause an antigen-antibody reaction. For the objective of the present application, antibody refers to the antibody that specifically binds to the biomarker for diagnosing CARAS of the present application. The antibody of the present application includes polyclonal antibody, monoclonal antibody and recombinant antibody. The above antibodies can be easily prepared by techniques known in the art. For example, polyclonal antibodies can be produced by methods known in the art including the process of injecting the above-mentioned biomarker protein antigen of CARAS into animals and taking blood from animals to obtain serum containing antibodies. This polyclonal antibody may be prepared from any animal such as goat, rabbit, sheep, monkey, horse, pig, cow and dog. Furthermore, monoclonal antibodies may be prepared by hybridoma method or phage antibody library technology known in the art. The antibody prepared by the above method may be separated and purified by gel electrophoresis, dialysis, salt precipitation, ion exchange chromatography, affinity chromatography, etc. Moreover, the antibody of the present application includes not only the complete morphology with two full-length light chains and two full-length heavy chains, but also the functional fragments of antibody molecules. A functional fragment of antibody refers to the fragment with at least antigen binding function, including Fab, F(ab′), F(ab′)2 and Fv, etc. Furthermore, the antibody of the present application may be obtained commercially.

The term “peptide nucleic acid” used in the present application refers to a synthetic polymer similar to deoxyribonucleic acid or ribonucleic acid. Deoxyribonucleic acid has a phosphoribosyl skeleton, but peptide nucleic acid has a repetitive N-(2-aminoethyl)-glycine skeleton connected by peptide bonds, Which results in much greater binding and stability for deoxyribonucleic acid or ribonucleic acid, making it applicable in molecular biology, diagnostic analysis, and antisense therapeutics.

In the present application, the “aptamer” is an oligonucleotide or peptide molecule.

Optionally, the primer pairs include a primer pair specifically bound to MGAT3, a primer pair specifically bound to miR-199a-5p, and a primer pair specifically bound to circ_0070934, where the primer pair specifically bound to MGAT3 includes sequences as shown in SEQ ID NO.1-2, the primer pair specifically bound to miR-199a-5p includes a sequence as shown in SEQ ID NO.5; and the primer pair specifically bound to circ_0070934 includes sequences as shown in SEQ ID NO.3-4.

Optionally, the sample includes biopsy, curettage, blood, urine, saliva, cell culture, mucous membrane samples, feces, intestinal lavage, joint fluid, cerebrospinal fluid, bile samples, respiratory secretions and bronchoalveolar lavage fluid samples, preferably blood.

Another aspect of the present application provides a system/device for diagnosing CARAS, including:

a detecting component, used for detecting the expression levels of biomarkers in the sample of the subject, where the biomarkers include cure 0070934, MGAT3 and miR-199a-5p; and

result judging component, used for outputting whether the subject suffers from CARAS or the risk of suffering from CARAS according the expression levels of the biomarkers detected by the detecting component.

Optionally, the result judging component includes an input module, an analysis module and an output module;

Optionally, the input module is used for inputting the expression level of the biomarkers detected by the detecting component.

Optionally, the analysis module is used for analyzing whether the subject suffers from CARAS or the risk of suffering from CARAS according to the expression level of the biomarkers.

Optionally, the output module is used for outputting an analysis result of the analysis module.

The subject described in the present application includes any animal and also refers to human and non-human animals; non-human animals include all vertebrates, for example, mammals such as non-human primates (especially higher primates), sheep, dogs, rodents (such as mice or rats), guinea pigs, goats, pigs, cats, rabbits, cattle, and any domestic animals or pets; and non-mammals such as chickens, amphibians, reptiles, etc. Preferably, the subject is a human.

Another aspect of the present application provides an application of a reagent for detecting the biomarkers provided in one aspect of the present application in preparing the system/device for diagnosing CARAS.

Another aspect of the present application provides a tool for diagnosing CARAS, where the tool includes the reagent for detecting the biomarkers provided in one aspect of the present application in a sample.

Optionally, the tool includes a kit, a chip and a test strip.

Another aspect of the present application provides a method for diagnosing a subject in terms of suffering from CARAS or a risk of suffering from CARAS.

Optionally, the method includes judging whether the subject suffers from CARAS or the risk of suffering from CARAS through a detection result of the expression level of biomarkers in the subject, where the biomarkers include circ_0070934, MOATS and miR-199A-5P;

Optionally, the method includes:

(1) collecting a sample of a subject;

(2) detecting the sample in terms of expression level of the biomarkers; and

(3) judging whether the subject suffers from CARAS or the risk of suffering from CARAS according to the detection result of the expression level of the biomarkers.

The present application achieves the following advantages and beneficial effects:

the present application is the first to detect circ_0070934/miR-199a-5p/MGAT3 levels in subjects with CARAS, and it is found that the expression of circ_0070934 and MGAT3 is down-regulated compared to that of healthy controls, while the expression level of miR-199a-5p is up-regulated; it is also found that these biomarkers may be used to effectively diagnose CARAS, and have the highest diagnostic performance for CARAS when the three biomarkers circ_0070934, miR-199a-5p and MGAT3 are used in combination.

### BRIEF DESCRIPTION OF THE DRAWINGS

FIG. 1 shows a statistical chart of protein expression level of serum MGAT3 in patients with combined allergic rhinitis and asthma syndrome (CARAS) and healthy controls detected by enzyme-linked immunosorbent assay (ELISA),

FIG. 2A-FIG. 2C show statistical charts of expression levels of circ_0070934, miR-199a-5p and MGAT3 in CARAS patients and healthy controls by quantitative real-time fluorescence polymerase chain reaction (PCR), in which FIG. 2A is the statistical chart of MGAT3, FIG. B is the statistical chart of miR-199a-5p and FIG. C is the statistical chart of circ_0070934.

FIG. 3A-FIG. 3D illustrate results of correlation analysis between circ_0070934/MGAT3 and clinical indicators of CARAS patients, in which FIG. 3A shows s relationship between MGAT3 mRNA and an absolute value of peripheral blood eosinophils, FIG. 3B shows a relationship between MGAT3 mRNA and a percentage of peripheral blood eosinophils, FIG. 3C shows a relationship between circ_0070934 and an absolute value of peripheral blood eosinophils, and FIG. 3D shows a relationship between circ_07099 and a percentage of peripheral blood eosinophils.

FIG. 4 shows analysis results of receiver operating characteristic (ROC) curves of MGAT3, miR-199a-5p and circ_0070934 as CARAS biomarkers.

FIG. 5 shows ROC curves analysis results of multi-factor combined diagnosis of MGAT3, miR-199a-5p and circ_0070934 as CARAS biomarkers.

### DETAILED DESCRIPTION OF THE EMBODIMENTS

The present application is described in further detail below in conjunction with the accompanying drawings and embodiments. The following embodiments are used only to illustrate the application and are not intended to limit the scope of the application. Experimental methods for which specific conditions are not indicated in the embodiments are generally in accordance with conventional conditions, or in accordance with the conditions recommended by the manufacturer.

### Embodiment 1 Screening of Biomarkers

I. Experimental Method

1. Research Object

In this study, from June 2020 to December 2021, 38 patients with combined allergic rhinitis and asthma syndrome (CARAS) and 43 healthy controls matched with gender and age are collected from the outpatient department of respiratory and critical care medicine of Changzhou No.2 People's Hospital affiliated to Nanjing Medical University. The diagnostic criteria of allergic rhinitis refer to the Chinese Guideline for diagnosis and treatment of allergic rhinitis [Cheng L., Chen J., Fu Q., et al. Chinese Society of Allergy Guidelines for Diagnosis and Treatment of Allergic Rhinitis [J]. Allergy Asthma Immunol Res. 2018, 10(4):300-53.DOI:10.4168/aair.2018.10.4.300.]; and asthma is diagnosed according to the Chinese Guidelines for bronchial asthma prevent and management (2020 edition) [Asthma group of Chinese Thoracic Society. [Guidelines for Bronchial Asthma Prevent and Management (2020 edition) Asthma Group of Chinese Thoracic Society] [J]. Chinese Journal of Tuberculosis and Respiratory Diseases. 2020, 43 (12): 1023-48, DOI: 1.0.3760/cma.j.cn12147-2020 06 18-00721.]. The control group includes healthy volunteers, with no history of asthma and allergic rhinitis and other allergic and immune system diseases. CARAS patients are newly diagnosed patients and have not subjected to anti-asthma drugs. All subjects are required to exclude diseases such as complicated infection, pulmonary embolism, chronic obstructive pulmonary disease, tuberculosis, blood system diseases and liver dysfunction, and all subjects are required to subject to examinations including blood routine, liver function, kidney function, blood sugar, blood lipid and electrocardiogram to exclude basic diseases. All subjects are required to sign the informed consent form, and this study is approved by the Ethics Committee of Changzhou No.2 People's Hospital affiliated to Nanjing Medical University.

2. Blood Samples Collection

Peripheral venous blood of 10 milliliters (mL) are collected by ethylene diamine tetraacetic acid (EDTA) anticoagulation tube from each patient and healthy control, and 5 mL of the peripheral venous blood is centrifuged at 3,000 revolutions per minute (rpm) for 10 minutes (min), with serum being separated and used to measure MGAT3 protein level by enzyme-linked immunosorbent assay (ELISA); and the remaining 5 mL of whole blood is used for quantitative real-time polymerase chain reaction (qRT-PCR) to determine the expression levels of circ_0070934, miR-199a-5p and MGAT3. Blood samples of all subjects are stored at −80 degree Celsius (° C.).

3. ELISA Determination

Human MGAT3 ELISA kit (Shanghai Kexing Biotechnology Co., Ltd.) is used to detect the serum samples of CARAS patients and control group in terms of MGAT3 content, and the experiment and operation are carried out strictly according to the instructions of the kit.

4. qRT-PCR Detection

Total RNA is extracted by RNAliquid overspeed whole blood total RNA extraction kit (Beijing HT-biotech Co., Ltd.), and the experimental steps are carried out according to its instructions. The extracted RNA is detected by ultraviolet spectrophotometer to determine concentration and purity, and reverse-transcribed by FastQuant cDNA first strand synthesis kit (Tiangen Biotech (Beijing) Co., Ltd.) to synthesize mRNA and cDNA/circRNA and cDNA, followed by miRNA cDNA reverse transcription using miRNA first strand cDNA synthesis (plus tail method) (Sangon Biotech (Shanghai) Co., Ltd.). mRNA and circRNA are amplified with SuperReal PreMix Plus (SYBR Green) (Tiangen Biotech (Beijing) Co., Ltd,), the miRNA is amplified by miRNA fluorescent quantitative PCR kit (dye method) (Sangon Biotech (Shanghai) Co., Ltd.), and the data are analyzed by ABI 7300 fluorescent quantitative PCR instrument and 2-ΔΔCT method. The internal references of circ0070934, MGAT3, and miRNA are GAPDH, GAPDH+ACTB, and U6 respectively; the upstream and downstream primers of MGAT3, circ_0070934, ACTB, GAPDH and the upstream primer of miR-199a-5p are synthesized by Beijing Biomed Company, and the upstream and downstream primers of L16 and the downstream primer of milt-199a-5p are universal primers provided by miRNA fluorescent quantitative PCR kit.

The primer sequences are as follows:

TABLE 1
Primer sequences

| | | | SEQ ID |
|---|---|---|---|
| Gene | Primer | Sequence | NO. |
| MGAT3 | Upstream primer | 5′-TCCTGTTTCCCTCACTGTGC-3′ | SEQ ID |
| | | | NO. 1 |
| | Downstream | 3′-ACACACGCACAAACATGAGC-5′ | SEQ ID |
| | primer | | NO. 2 |
| circ_ 0070934 | Upstream primer | 5′-GGTGAAAGGACTGATCAACCAT-3′ | SEQ ID |
| | | | NO. 3 |
| | Downstream | 3′-TGTCTTGAGCTTTCCTGCCT-5′ | SEQ ID |
| | primer | | NO. 4 |
| miR-199a-5p | Upstream primer | 5′-CCCAGTGTTCAGACTACCTGTTC-3′ | SEQ ID |
| | | | NO. 5 |
| ACTB | Upstream primer | 5′-TCCGCAAAGACCTGTACGC-3′ | SEQ ID |
| | | | NO. 6 |
| | Downstream | 3′-CTGGAAGGTGGACAGCGAG-5′ | SEQ ID |
| | primer | | NO. 7 |
| GAPDH | Upstream primer | 5′-TCGACAGTCAGCCGCATCTTCTTT- | SEQ ID |
| | | 3′ | NO. 8 |
| | Downstream | 3′-ACCAAATCCGTTGACTCCGACCTT- | SEQ ID |
| | primer | 5′ | NO. 9 |

The downstream primers of internal reference U6 and miR-199a-5p are provided by miRNA fluorescent quantitative PCR kit.

5. Statistical Analysis

SPSS 21.0 statistical software is used for analysis, with measurement data tested for normality using the Kolmogorov-Smirnov test, and the measurement data conforming to a normal distribution are expressed as mean±standard deviation, those not conforming to the normal distribution are expressed as median (quartiles), and categorical variable data are expressed as proportions. Two samples are compared using the t test (normally distributed data) or the Mann-Whitney U test (non-normally distributed data), and correlations are determined by Pearson correlation analysis (normally distributed data) or Spearman rank correlation analysis (non-normally distributed data). The receiver operating characteristic (ROC) curve is used to determine the diagnostic sensitivity and specificity, and the area under the curve (AUC) is used to determine the diagnostic effectiveness, with AUC=0.5 indicating no diagnostic value, and the closer the AUC is to 1, the more accurate the diagnosis is, and P<0.05 indicates a statistically significant difference (two-tailed method).

II. Experimental Results

1. Clinical Characteristics of the Research Object.

In this study, 38 patients with CARAS and 43 healthy controls with matching age, gender, and body weight index in both groups are included; compared with healthy controls, lung function FEV₁% and FEV₁/FVC in CARAS patients are significantly lower (P<0.05), while the absolute eosinophil value, eosinophil percentage and nitric oxide (FeNO) inexhaled breath are significantly higher (P<0.001) than those in the healthy controls (see Table 2).

TABLE 2
Clinical characteristics of subjects

| Groups | CARAS (n = 38) | | Control (n = 43) | | P |
|---|---|---|---|---|---|
| Gender | 22/16 | | 27/16 | | 0.653 |
| (male/female) | | | | | |
| Age (Y) | 40.73 ± 13.65 | | 40.30 ± 10.40 | | 0.738 |
| BMI (Kg/m²) | 25.30 ± 3.46 | | 26.43 ± 3.51 | | 0.606 |
| FEV₁% | 88.19 ± 27.27 | | 108 ± 8.67 | | 0.024 |
| FEV₁/FVC (%) | 73.55 | (67.13-82.00) | 83.00 | (97.70-114.10) | 0.016 |
| EOS (×10⁹/L) | 0.36 | (0.20-0.71) | 0.12 | (0.07-0.21) | <0.001 |
| EOS % | 6.00 ± 4.38 | | 1.80 ± 1.33 | | <0.001 |
| FeNO (ppb) | 61.00 | (26.50-93.50) | 19.00 | (14.00-27.00) | <0.001 |

2. Serum Expression Levels of MGAT3 in CARAS Patients and Healthy Controls.

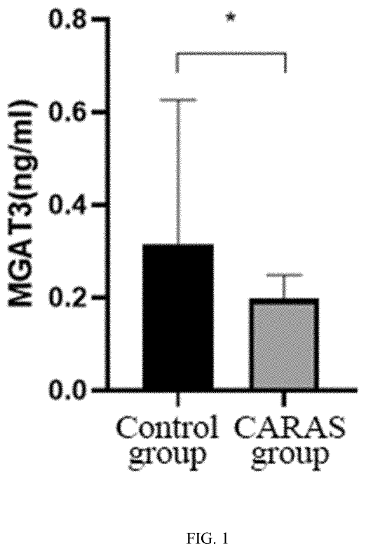

The results of ELISA experiments indicate that the protein expression of MGAT3 in peripheral blood serum of CARAS patients (0.20±0.05) is significantly decreased (P=0.035) compared to that of controls (0.32±0.31) (see FIG. 1).

3. Expression Levels of Circ_0070934, miR-199a-5p and MOATS in CARAS Patients and Healthy Controls

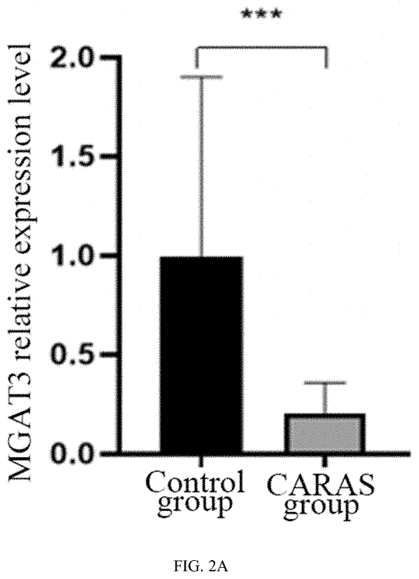

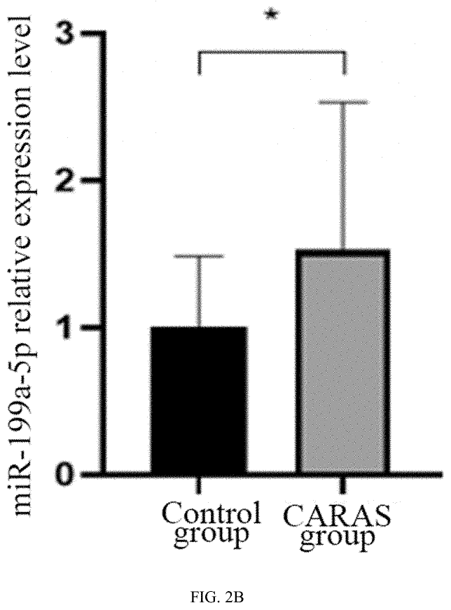

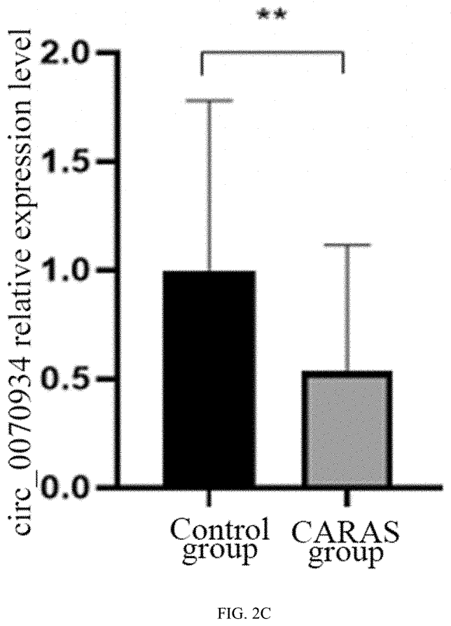

The results of qRT-PCR show that. MGAT3 expression is significantly lower in CARAS patients compared with controls (FIG. 2A, P<0.001) and circ_0070934 expression is also significantly lower in CARAS patients (FIG. 2C, P=0.001), while miR-199a-5p expression is significantly upregulated (FIG. 2B, P=0.013).

4. Correlations Between Circ_0070934/MGAT3/miR-199a-5p and Clinical Indicators of CARAS Patients

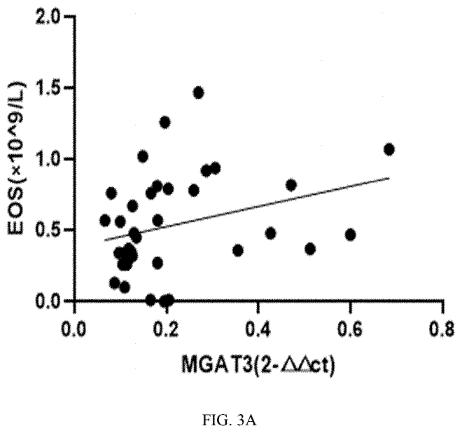

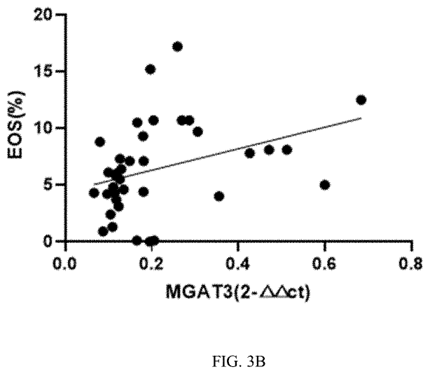

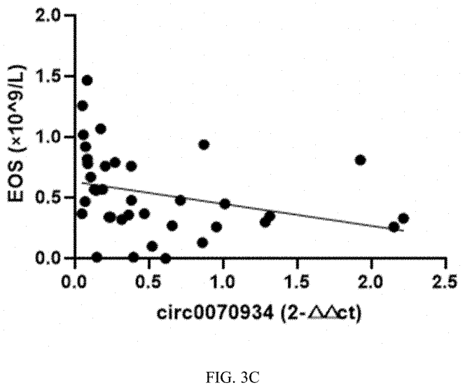

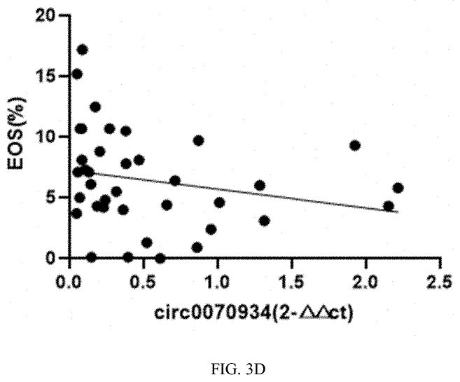

The level of MGAT3 mRNA in peripheral blood of CARAS patients is positively correlated with the absolute value of eosinophils (70.412, P=0.011) and the percentage of eosinophils (r=0.469, P=0.003), while circ_0070934 is negatively correlated with absolute eosinophil (r=−0.516, P=0.001) and eosinophil percentage (r=−0.348, P=0.035) (Table 3, FIGS. 3A-D). MGAT3 and circ_0070934 have no correlation with FeNO, FEV₁% and FEV₁/FVC; and there is no significant correlation between miR-199a-5p and absolute value of eosinophils, percentage of eosinophils, FeNO, FEV₁% and FEV₁/FVC (Table 3).

TABLE 3
Correlation between circ_0070934/miR-199a-5p/MGAT3
and clinical indicators of CARAS patients

| | | EOS | | FeNO | | FEV₁/FVC |
|---|---|---|---|---|---|---|
| | | (×10⁹/L) | EOS % | (ppb) | FEV₁ % | (%) |
| MGAT3 | r | 0.412 | 0.469 | 0.268 | 0.025 | −0.060 |
| | P | 0.011 | 0.003 | 0.132 | 0.884 | 0.772 |
| miR-199a-5p | r | 0.261 | 0.236 | 0.339 | 0.123 | 0.101 |
| | P | 0.118 | 0.159 | 0.053 | 0.469 | 0.552 |
| circ0070934 | r | −0.516 | −0.348 | −0.139 | −0.005 | 0.029 |
| | P | 0.001 | 0.035 | 0.441 | 0.974 | 0.864 |

### Embodiment 2 Diagnostic Efficacy Analysis

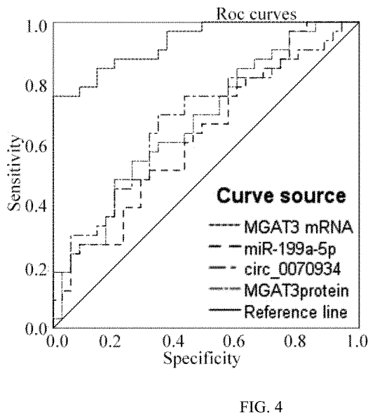

The ROC curve analysis is performed to evaluate the effectiveness of MGAT3, miR-199a-5p and circ0070934 as CARAS biomarkers (Table 4, FIG. 4). When the cut-off value is 0.208, the sensitivity of MGAT3 mRNA as a biomarker of CARAS is 73.7%, the specificity is 1, and the AUC is 0.933 (95%0=0.882-0.984, P<0.001), a best diagnostic performance as a biomarker of CARAS.

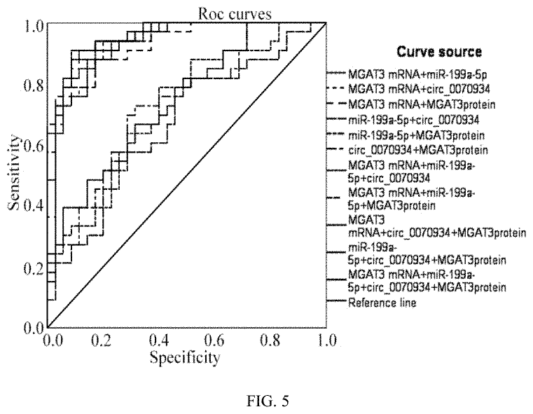

ROC curves analysis of multi-factor combined diagnosis (Table 4, FIG. 5) shows that when the cut-off value is 0.539, the sensitivity, specificity and AUC of MGAT3, miR-199a-5p and circ_0070934 as biomarkers for combined diagnosis of CARAS are 92.1%, 93% and 0.959 respectively (95% CI=0.916-L000, P<0.001)

TABLE 4
Effectiveness of MGAT3, miR-199a-5p and circ0070934 as CARAS biomarkers

| | | | | | 95% | |
|---|---|---|---|---|---|---|
| | Cut-off | | | | confidence | |
| | value | Sensitivity | Specificity | AUC | interval | P |
| MGAT3 mRNA | 0.208 | 73.7% | 1 | 0.933 | 0.882-0.984 | <0.001 |
| miR-199a-5p | 1.166 | 55.3% | 72.1% | 0.661 | 0.543-0.779 | 0.013 |
| circ_0070934 | 0.620 | 71.1% | 69.8% | 0.717 | 0.604-0.829 | 0.001 |
| MGAT3 protein | 0.191 | 54.5% | 74.3% | 0.673 | 0.545-0.800 | 0.014 |
| MGAT3 mRNA + miR-199a-5p | 0.557 | 89.5% | 88.4% | 0.946 | 0.874-0.994 | <0.001 |
| MGAT3 mRNA + circ_0070934 | 0.423 | 92.1% | 83.7% | 0.944 | 0.885-0.989 | <0.001 |
| MGAT3 mRNA + MGAT3 protein | 0.761 | 75.8% | 97.1% | 0.932 | 0.877-0.988 | <0.001 |
| miR-199a-5p + circ_0070934 | 0.389 | 84.2% | 53.5% | 0.722 | 0.542-0.800 | 0.015 |
| miR-199a-5p + MGAT3 protein | 0.454 | 78.8% | 60.0% | 0.720 | 0.600-0.841 | 0.002 |
| Circ_0070934 + MGAT3 | 0.521 | 72.7% | 68.6% | 0.736 | 0.619-0.853 | 0.001 |
| protein | | | | | | |
| MGAT3 mRNA + miR-199a- | 0.539 | 92.1% | 93.0% | 0.959 | 0.891-1.000 | <0.001 |
| 5p + circ_0070934 | | | | | | |
| MGAT3 mRNA + miR-199a- | 0.585 | 87.9% | 88.6% | 0.939 | 0.886-0.993 | <0.001 |
| 5p + MGAT3 protein | | | | | | |
| MGAT3 mRNA + | 0.443 | 93.9% | 82.9% | 0.943 | 0.894-0.992 | <0.001 |
| circ_0070934 + MGAT3 protein | | | | | | |
| miR-199a-5p + | 0.529 | 66.7% | 68.6% | 0.740 | 0.625-0.856 | 0.001 |
| circ_0070934 + MGAT3 protein | | | | | | |
| MGAT3 mRNA + miR-199a-5p + | 0.486 | 90.9% | 88.6% | 0.948 | 0.900-0.996 | <0.001 |
| circ_0070934 + MGAT3 protein | | | | | | |

The preferred embodiments of this application are described in detail with the above drawings, but this application is not limited to the specific details of the above embodiments, Within the technical concept of this application, many simple modifications may be made to the technical scheme of this application, and these simple modifications all fall into the protection scope of this application.

In addition, it should be noted that the specific technical features described in the above specific embodiments can be combined in any suitable way without contradiction. In order to avoid unnecessary repetition, this application will not separately explain various possible combinations.

In addition, any combination of different embodiments of this application may also be made, and as long as it does not violate the idea of this application, it should also be regarded as the content disclosed in this application.

What is claimed is:

1. Biomarkers for diagnosing combined allergic rhinitis and asthma syndrome (CARAS), comprising circ_0070934, MGAT3, and miR-199a-5p.

2. An application of reagents for detecting the biomarkers according to claim 1 in a sample for preparing diagnostic tools of CARAS.

3. The application according to claim 2, wherein the reagents comprise reagents for detecting expression levels of the biomarkers in the sample by sequencing technology, nucleic acid hybridization technology, nucleic acid amplification technology and protein immunization technology.

4. The application according to claim 2, wherein the reagents comprise:
primer pairs, probes or antisense nucleotides specifically combined with genes of the biomarkers; and
antibodies, interacting proteins, ligands, nanoparticles or aptamers specifically bound to proteins or peptide fragments of the biomarkers.

5. The application according to claim 4, wherein the primer pairs comprise a primer pair specifically bound to MGAT3, a primer pair specifically bound to miR-199a-5p, and a primer pair specifically bound to circ_0070934;
wherein the primer pair specifically bound to MGAT3 comprises sequences as shown in SEQ ID NO.1-2; the primer pair specifically bound to miR-199a-5p comprises a sequence as shown in SEQ ID NO.5; and the primer pair specifically bound to circ_0070934 comprises sequences as shown in SEQ ID NO.3-4.

6. The application according to claim 2, wherein the sample comprises biopsy, curettage, blood, urine, saliva, cell culture, mucous membrane samples, feces, intestinal lavage, joint fluid, cerebrospinal fluid, bile samples, respiratory secretions and bronchoalveolar lavage fluid samples.

7. A system/device for diagnosing CARAS, comprising:
a detecting component, used for detecting expression levels of biomarkers in a sample of a subject, wherein the biomarkers comprise circ_0070934, MGAT3 and mir-199a-5p; and
result judging component, used for outputting whether the subject suffers from CARAS or a risk of suffering from CARAS according to the expression levels of the biomarkers detected by the detecting component; wherein
the result judging component comprises an input module, an analysis module and an output module;
the input module is used for inputting the expression levels of the biomarkers detected by the detecting component;
the analysis module is used for analyzing whether the subject suffers from CARAS or the risk of suffering from CARAS according to the expression levels of the biomarkers; and
the output module is used for outputting an analysis result of the analysis module.


SEQUENCE LISTING (9 sequences)

SEQ ID NO: 1 - DNA, 20 bp, synthetic construct
  source 1..20: mol_type = other DNA
tcctgtttccctcactgtgc

SEQ ID NO: 2 - DNA, 20 bp, synthetic construct
  source 1..20: mol_type = other DNA
acacacgcacaaacatgagc

SEQ ID NO: 3 - DNA, 22 bp, synthetic construct
  source 1..22: mol_type = other DNA
ggtgaaaggactgatcaaccat

SEQ ID NO: 4 - DNA, 20 bp, synthetic construct
  source 1..20: mol_type = other DNA
tgtcttgagctttcctgcct

SEQ ID NO: 5 - DNA, 23 bp, synthetic construct
  source 1..23: mol_type = other DNA
cccagtgttcagactacctgttc

SEQ ID NO: 6 - DNA, 19 bp, synthetic construct
  source 1..19: mol_type = other DNA
tccgcaaagacctgtacgc

SEQ ID NO: 7 - DNA, 19 bp, synthetic construct
  source 1..19: mol_type = other DNA
ctggaaggtggacagcgag

SEQ ID NO: 8 - DNA, 24 bp, synthetic construct
  source 1..24: mol_type = other DNA
tcgacagtcagccgcatcttcttt

SEQ ID NO: 9 - DNA, 24 bp, synthetic construct
  source 1..24: mol_type = other DNA
accaaatccgttgactccgacctt